(19) United States
(12) Patent Application Publication
(10) Pub. No.: US 2004/0142860 A1
(43) Pub. Date: Jul. 22, 2004

(54) Central nervous system-derived immune privilege factor and uses thereof

(75) Inventors: Michal Eisenbach-Schwartz, Rehovot (IL); Pierre Beserman, Moshav Satarya (IL); David L. Hirschberg, Menlo Park, CA (US)

Correspondence Address: BROWDY AND NEIMARK, P.L.L.C., 624 NINTH STREET, NW, SUITE 300, WASHINGTON, DC, 20001-5303, US

(73) Assignee: Yeda Research and Development Co. Ltd., Rehovot (IL)

(21) Appl. No.: 09/814,699

(22) Filed: Mar. 23, 2001

Related U.S. Application Data
(63) Continuation of application No. 09/261,369, filed on Mar. 3, 1999, now abandoned.
Application No. 09/261,369 is a continuation of application No. PCT/IL97/00294, filed on Sep. 3, 1997.
(60) Provisional application No. 60/025,376, filed on Sep. 3, 1996.

(51) Int. Cl. A61K 38/17; C07K 14/74

(52) U.S. Cl. 514/12; 530/350

(57) ABSTRACT

The present invention is directed to a central nervous system-derived heat stable immune privilege factor which exerts an inhibitory effect on macrophage migration and/or macrophage phagocytic activity. In addition, the factor exerts an inhibitory effect on the ability of macrophages and T cells to adhere to extracellular matrix and/or fibronectin. The invention is also directed to the isolation and methods for use of this immune privilege factor for the inhibition of inflammation in the central nervous system generally and at specific lesions in the central nervous system.

The present application is a continuation of copending application Ser. No. 09/261,369 filed Mar. 8, 1999, which is a continuation of International Patent Application PCT/IL97/00294, filed Sep. 3, 1997, which International Application claims priority benefits to United States Provisional Application No. 60/025,376 filed Sep. 3, 1996, each of which is incorporated by reference herein in its entirety.

### 1. FIELD OF THE INVENTION

The present invention is directed to a central nervous system (CNS)-derived heat stable immune privilege factor (IPF). The present invention is also directed to methods for the use of the factor in the modulation of immune responses, including, but not limited to, inhibiting inflammation caused by disease in the central nervous system.

### 2. BACKGROUND OF THE INVENTION

Citation or Identification of any reference in Section 2 or any other section of this application shall not be construed as an admission that such reference is available as prior art to the present invention.

The environment surrounding the axons in the CNS and peripheral nervous system (PNS) of mammals is inhibitory for neuronal growth in the adult animal. After injury, the neurons in the peripheral nervous system are able to regenerate their axons, but no regeneration occurs in the CNS. Recently, Lotan and Schwartz (Lotan and Schwartz, 1994, FASEB 8:1026-1033), have proposed that axonal regeneration is affected by the inflammatory response and the surrounding environment which is composed of the various glial cells such as oligodendrocytes, astrocytes, and microglia, as well as their soluble extracellular matrix (ECM) products in the CNS and Schwann cells and their soluble ECM products in the PNS. The environment in both the PNS and CNS also includes cells of the immune system, such as macrophages, which are known to invade the PNS and CNS after injury, as well as the various cytokines associated with these immune system-derived cells.

Both the CNS and PNS environment are inhibitory for growth of adult neurons under normal circumstances. However, following injury to PNS neurons, the PNS environment is somehow modified to allow axonal regeneration of the injured PNS neurons to occur. On the other hand, in the mammalian CNS it appears that such regeneration-associated modifications in the environment fail to occur and thus, axonal regeneration of injured CNS neurons does not occur. It has been proposed that the cells of the immune system play a role in the modification of the neuronal environment (see Lotan and Schwartz, Id.).

A classical inflammatory response is characterized by the invasion of myelomonocytic cells into the afflicted tissue within hours after injury. Among these early invaders are macrophages capable of mediating a myriad of functions, from removal of debris and dead and dying tissue by phagocytosis to secretion of enzymes and growth factors that facilitate tissue regeneration. Macrophage-derived cytokines, such as platelet-derived growth factor (PDGF), tumor necrosis factor alpha (TNFα), transforming growth factor beta (TGFβ1), heparin-binding epidermal growth factor (HB-EGF), interleukin-1 (IL-1) and interleukin-6 (IL-6), have been shown to have secondary effects on other bone marrow derived cells and on resident cells in the injured tissue. In the CNS and PNS, macrophage-derived cytokines have been shown to increase the level of secondary cytokines and factors needed for regenerative growth such as nerve growth factor (NGF), cell adhesion molecules (CAMs), and ECM components such as heparinase.

There is evidence supporting the idea that differences in macrophage response to injury in the nervous system can affect regenerative outcome. Following nerve injury, the inflammatory response, including Wallerian degeneration, is dependent upon macrophages. If macrophage invasion is blocked in the sciatic nerve by use of Boyden chambers which isolate the sciatic nerve tissue from the circulatory system, degeneration and subsequent regeneration is greatly impaired. C57/6/01a mice have a defect in macrophage recruitment and after sciatic crush, Wallerian degeneration in these mice occurs very slowly compared to that in mice with normal macrophage recruitment, (Lunn et al., 1990, Neuroscience 35:157-165). Further, subsequent regeneration of the sciatic nerve in these mice is very slow and not complete.

In lower vertebrates, for example, fish such as Cyprinus carpio (carp), in which CNS regeneration occurs successfully, macrophages are constitutively present in the optic nerve (a CNS nerve) and after injury are associated with a decrease in the number of oligodendrocytes in cultures of crushed fish optic nerve. If invasion of these macrophages is prevented, larger numbers of oligodendrocytes are observed. In addition, the appearance of these macrophages is concurrent with the production of soluble substances that are cytotoxic to both fish and rat oligodendrocytes in vitro (Cohen et al., 1990, Brain Res. 537:24-32; Sivron et al., 1990, Glia 3:267-276). These same macrophage-associated factors can facilitate regeneration when applied to mammalian CNS in vivo (Schwartz et al., 1985, Science 228:600-603; Lavie et al., 1987, Brain Res. 419:166-172; Lavie et al., 1990, J. Comp. Neurol. 298(3):293-314).

Moreover, fish optic nerve cultures contain lower numbers of oligodendrocytes than rat optic nerve cultures following axonal injury. The lower oligodendrocyte number in fish may be a result of invading blood-derived macrophages. If the invasion is blocked, high numbers of oligodendrocytes are found in organ culture (Sivron et al., 1990, Glia 3:267-276; Sivron et al., 1991, Glia 4:591-601). Therefore, the context of interaction between the immune system and the nervous system may have a strong impact on whether regeneration will occur such that the appearance of macrophage's at the site of nerve injury is critical for nerve growth and regeneration at the site of injury.

Therefore, the limited number of macrophages at the site of nerve injury in the central nervous system of higher vertebrates may be due to an inhibition of macrophage recruitment to these injured sites.

Several factors are known which modulate macrophage activity. For example, tuftsin, a derivative of IgG, is a potent macrophage stimulator. Interferon-γ and Tumor Necrosis Factor are also potent stimulators. There are also factors which inhibit macrophage activity, called MIFs. For example, a tripeptide, Thr-Lys-Pro, TKP, a derivative of tuftsin, has been shown to inhibit macrophage migration and reduce secretion of IL-1 macrophages (see Nishioka et al., 1973, Biochem. Biophys. Acta 310:217-228; Bump et al., 1990, Mol. Cell, Biochem. 92:77-84; Fridkin et al., 1989, Crit. Rev. Biochem. Mol. Bio. 24:1-40; Tzehoval et al., 1978. Proc. Natl. Acad. Sci. USA 75:3400-3404; Thanos et al., 1993, J. Neurosci. 13:455-466; Plata-Salaman, 1989, Brain Behav. Immunol. 3:193-213; Wagle et al., 1989, Biochem. Biophys. Rev. Commun. 159:1147-1153; Sienion et al., 1991, Arch. Immunol. Ther. Exp. 39:605-611; Auriault et al., 1985, Immunopharmac. 7:73-79). Another MIF is Tolrestat, an aldose reductase inhibitor (Calcott et al., 1994, Exp. Neurol. 128:226-232).

Thanos et al. (Thanos et al., 1993, J. Neurosci. 13:455-466) showed that single or repeated injections of TKP into the vitreous body during and after transection of the optic nerve resulted in the retardation of axotomy-induced ganglion cell degradation in the retina.

### 3. SUMMARY OF THE INVENTION

The present invention is directed to a composition which comprises a heat stable immune privilege factor (IPF) which has anti-inflammatory activity. The present invention is based, at least in part, on the discovery that nerve tissue of the central nervous system, including optic nerve and brain tissue, contains a factor of approximately 350 Daltons which exhibits inhibitory activity on macrophage migration and on macrophage phagocytic activity. The factor also inhibits the ability of macrophages and T cells to adhere to extracellular matrix and fibronectin. The immune privilege factor can be isolated from the central nervous system tissue itself or, in a preferred embodiment, from cell culture medium or buffer which has been conditioned by growing or placing the central nervous system tissue in the medium or buffer for a period of time. In a more preferred embodiment, IPF can be further isolated by subjecting the conditioned medium or buffer to gel filtration chromatography. In yet another preferred embodiment the immune privilege factor can be purified by subjecting the conditioned medium or buffer to gel filtration chromatography followed by reverse phase high pressure liquid chromatography (HPLC) and then by thin layer chromatography (TLC) or ion exchange column chromatography. The composition is used as an inhibitor of macrophage migratory and phagocytic activity and inflammation in animals, preferably mammals, including humans. The composition is also used as an inhibitor of macrophage and T cell adhesive activity in animals, preferably mammals, including humans.

The present invention is also directed to a composition comprising the immune privilege factor which further comprises a pharmaceutically acceptable carrier. The pharmaceutical composition is used as an inhibitor of macrophage migration and/or macrophage phagocytic activity and inflammation in animals, preferably mammals, including humans. The pharmaceutical composition is also used as an inhibitor of macrophage and T cell adhesive activity in animals, preferably mammals, including humans.

The present invention is also directed to methods of use of the immune privilege factor for the inhibition of inflammation at a desired site. The method comprises applying an effective amount of central nervous system-derived immune privilege factor to a site to inhibit inflammation at the site. In a preferred embodiment, an effective amount of a therapeutic composition comprising the immune privilege factor and a pharmaceutical carrier is applied to a site to inhibit inflammation at the site. In another preferred embodiment, the method comprises applying an effective amount of central nervous system-derived immune privilege factor to a site of nerve injury in the central nervous system to inhibit inflammation.

Inflammatory diseases or conditions or disorders contributing to or caused by nerve injury for which the immune privilege factor of the present invention can be used to inhibit unwanted and dangerous inflammation in the central nervous system and eye are, for example and not by way of limitation, blunt trauma, AIDS-related dementia complex, HIV-related encephalopathy, post-polio syndrome, multiple sclerosis, myelitis, encephalitis, meningitis, rheumatic fever, complications and side-effects due to neurosurgery, subacute sclerosing panencephalitis, Huntington's disease, Devic's disease, Parkinson's disease, Sydenham chorea, posterior uveitis, anterior uveitis, sympathetic ophthalmia, retinitis, cystoid macular edema, optic neuritis, proliferative vitreoretinophathy, retinitis pigmentosa, glaucoma or a complication and/or side-effect from transplantation surgery or treatment of Parkinson's disease. In addition, it is envisioned that the present immune privilege factor (IPF) can be used to alleviate any conditions in which there is degeneration of the CNS, including the brain and the retina of the eye.

### 4. BRIEF DESCRIPTION OF THE FIGURES

FIGS. 1A-C are inverted fluorescence micrographs comparing the migration of macrophages towards different nerve types in culture medium. FIG. 1A shows migration towards optic nerve; FIG. 1B, towards sciatic nerve; FIG. 1C, control medium only. Arrows indicate macrophages. See Section 6.1 for details.

FIG. 2 is a bar graph showing the number of macrophages which migrated towards optic nerves or sciatic nerves. Cell culture medium (Medium) served as a control. Open bars represent three hour incubation before quantitation; solid black bars represent twenty-four incubation before quantitation.

FIG. 3 is a bar graph showing the number of macrophages which migrated towards optic nerve conditioned medium or sciatic nerve conditioned medium. Cell culture medium (Medium) served as a control. Open bars represent three hour incubation before quantitation; solid black bars represent twenty-four incubation before quantitation.

FIG. 4 depicts the effect of diluting the optic nerve conditioned medium or the sciatic nerve conditioned medium on macrophage migration. ●—sciatic nerve conditioned medium; ▪—optic nerve conditioned medium.

FIGS. 5A-C are photographs illustrating the difference in morphology between macrophages incubated in optic nerve conditioned medium ONCM (FIG. 5A); macrophages incubated in sciatic nerve conditioned medium, SNCM (FIG. 5B); and macrophages incubated in control medium (FIG. 5C).

FIG. 6 is a bar graph which demonstrates the ability of optic nerve conditioned medium (ON CM) to block the activity of sciatic nerve conditioned medium (SU CM) to induce macrophage migration towards sciatic nerve conditioned medium. See Section 7 for details.

FIG. 7 is a bar graph showing that the immune privilege factor of the present invention is found in the same elution fractions whether derived from optic nerve (ONCM f7 4-7+SNCM) or brain tissue (BCM f7 4-7+SNCM). CON, control; SNCM, sciatic nerve conditioned medium.

FIG. 8 is a bar graph showing that the immune privilege factor inhibits macrophage phagocytic activity. Abbreviations: Con, control; ONCM, optic nerve conditioned medium; SNCM, sciatic nerve conditioned medium. See text, Section 9, for details.

FIG. 9 is a bar graph showing that the immune privilege factor in optic nerve conditioned medium is heat resistant. Abbreviations: CON, control medium; bCON, boiled control medium; ONCM, optic nerve conditioned medium; bONCM, boiled optic nerve conditioned medium; SNCM, sciatic nerve conditioned medium; bSNCM, boiled sciatic nerve conditioned medium. See Section 10.1 for details.

FIG. 10 is a bar graph showing that the immune privilege factor in brain tissue conditioned medium is sensitive to protease treatment. Abbreviations: Con, control medium; SNCM, sciatic nerve conditioned medium; Brain-IPF, brain tissue conditioned medium; Brain-IPF K, brain tissue conditioned medium treated with Proteinase K. See Section 10.2 for details.

FIGS. 11A-C are graphs which demonstrate the immune privilege factor found in optic nerve conditioned medium has a molecular weight of approximately 350 Daltons. FIG. 11A is a bar graph illustrating the ability of optic nerve conditioned medium (ONCM) to block the ability of sciatic nerve conditioned medium (SNCM) to induce macrophage migration. FIG. 11B is an elution profile of the macrophage inhibitory activity found in ONCM. ONCM was fractionated over a gel filtration chromatography column and fractions were tested for the ability to inhibit N-formyl-Met-Leu-Phe (N-f-MLP), a macrophage chemoattractant. FIG. 11C is a standard curve for determining the molecular weight of the activity eluted off the column. The curve was calculated using bovine serum albumin (BSA), 10 amino acid peptides and tryptophan (Trp). See text for details.

FIG. 12 demonstrates the ability of optic nerve conditioned medium (ONCM) to inhibit tuftsin-induced macrophage migration.

FIG. 13 is a bar graph showing macrophage migration inhibitory activity of immune privilege factor after purification by gel filtration liquid chromatography, HPLC and TLC. Abbreviations: Con, control medium; SNCM, sciatic nerve conditioned medium; Brain-IPF, IPF purified from brain tissue conditioned medium; Brain-IPF K, IPF purified from brain tissue conditioned medium treated with Proteinase K; Optic nerve-IPF, IPF purified from optic nerve conditioned medium; optic nerve-IPF K, IPF purified from optic nerve conditioned medium treated with Proteinase K.

FIG. 14 is a graph of an elution profile showing that the immune privilege factor can be purified by ion exchange column chromatography and elutes off the column at 10 minutes at/with approximately 100 mM NaCl. See text, Section 13, for details.

FIG. 15 is a bar graph showing the ability of rat-derived immune privilege factor to inhibit the adhesive ability of human macrophages, thus demonstrating both inhibition of adhesion, which is a prerequisite for inflammation and cross-species reactivity. Abbreviations: Con, control medium; PMA, phorbol 12-myristate-13-acetate; Brain-IPF+PMA, brain-derived immune privilege factor with phorbol 12-myristate-13-acetate.

FIG. 16 is a bar graph showing the ability of rat-derived immune privilege factor to inhibit the adhesive ability of human T cells, thus demonstrating cross-species reactivity and a general effect on immune cells, i.e., IPF affects T cells as well as macrophages. Abbreviations: PMA, phorbol 12-myristate-13-acetate; Brain f7 4-7 b2+PMA, brain tissue conditioned medium-derived immune privilege factor purified by gel filtration liquid chromatography and HPLC batch 2 with 25 μg PMA; Brain f7 4-7 b2 K+PMA, brain tissue conditioned medium-derived immune privilege factor purified by gel filtration liquid chromatography and HPLC batch 2 with 25 μg PMA treated with Proteinase K; Brain f7 4-7 b1+PMA, brain tissue conditioned medium-derived immune privilege factor purified by gel filtration liquid chromatography and HPLC batch 1 with 25 μg PMA; Brain f7 4-7 b1 K+PMA, brain tissue conditioned medium-derived immune privilege factor purified by gel filtration liquid chromatography and HPLC batch 1 with 25 μg PMA treated with Proteinase K; ONCM f7 4-7 b2+PMA, optic nerve conditioned medium-derived immune privilege factor purified by gel filtration liquid chromatography and HPLC batch 2 with 25 μg PMA; ONCM f7 4-7 b2 K+PMA, optic nerve conditioned medium-derived immune privilege factor purified by gel filtration liquid chromatography and HPLC batch 2 with 25 μg PMA treated with Proteinase K.

FIG. 17 is a bar graph showing the effect of IPF on fas receptor expression in T cells as measured by the amount of fas receptor transcript. Abbreviations: SNCM, sciatic nerve conditioned medium; IPF+SNCM, immune privilege factor and sciatic nerve conditioned medium; IPF, immune privilege factor; and no treat., control where RPMI only was added to the cells. See text, Section 15, for details.

### 5. DETAILED DESCRIPTION OF THE INVENTION

5.1. Isolation and Identification of a Central Nervous System-Derived Factor Which Inhibits Macrophage Migration

The present invention is directed to a composition which comprises a heat stable immune privilege factor (IPF) which has anti-inflammatory activity. The anti-inflammatory activity of the factor is assessed by the inhibitory effect the factor has on macrophage migration and/or phagocytosis and/or on the adhesion of macrophages or T cells to extracellular matrix or fibronectin. The present invention is based, at least in part, on the surprising discovery that nerve tissue of the central nervous system, such as optic nerve and brain tissue, contains an immune privilege factor of approximately 350 Daltons which has macrophage migration and/or phagocytic inhibitory activity. In addition, the immune privilege factor has the ability to inhibit macrophage and T cell adhesion to extracellular matrix and fibronectin.

The immune privilege factor of the present invention is obtained from central nervous system tissue or from central nervous system tissue conditioned medium. The conditioned medium is produced by incubating a segment of central nervous system tissue, such as optic nerve or brain tissue, in cell culture medium or buffer for a period of time, removing the tissue, and filtering the medium or buffer, thus forming sterilized conditioned medium. The conditioned medium can be stored at −70° C. for up to a year without losing macrophage migration inhibitory activity.

The immune privilege factor can be further purified by subjecting the sterile conditioned medium to gel filtration chromatography, including size exclusion chromatography. Other methods for purification include, ion-exchange chromatography, hydrophobic interaction chromatography and affinity chromatography. For example and not by way of limitation, the conditioned medium, produced by incubation of a segment of optic nerve in phosphate buffered saline for one hour and then filter sterilization, is subjected to gel filtration chromatography on a SUPEROSE™ 12 (a gel filtration medium, Pharmacia, Uppsala, Sweden) column with PBS diluted 1:3 as the running buffer. Fractions are collected and each fraction is subjected to an in vitro assay to test for, e.g., inhibition of macrophage migration and/or phagocytic activity, and/or macrophage and/or T cell adhesion ability.

The collected fractions which contain the immune privilege factor isolated by chromatography can be subjected to further purification by, for example, reverse phase high pressure liquid chromatography (HPLC) and thin layer chromatography (TLC). Each of the collected fractions from HPLC and/or TLC is subjected to an in vitro assay to test for, e.g., inhibition of macrophage migration and/or phagocytic activity. The fractions can be also tested for inhibition of macrophage and/or T cell adhesion ability.

For example, one such in vitro assay uses modified Boyden chambers wherein the bottom chamber contains central nervous system tissue conditioned medium separated from the upper chamber by a filter. The upper chamber contains macrophages isolated from blood or derived from tissue culture. If the conditioned medium contains an inhibitor of macrophage migration then fewer macrophages will adhere to the filter separating the two halves of the Boyden chamber as compared to a control. The control, for example and not by way of limitation, can be sterile medium. In this manner it can be determined which fraction contains the immune privilege factor of the present invention.

The immune privilege factor of the present invention is a peptide/protein factor of approximately 350 Daltons. After the peptide/protein factor is isolated, for example, by gel filtration chromatography, HPLC and/or TLC, the peptide/protein factor can be further purified by standard methods including but not limited to ion exchange chromatography, affinity chromatography, centrifugation, differential solubility, or by any other standard technique for the purification of peptides or proteins. Ion exchange column chromatography is particularly suitable for purification of the immune privilege factor.

On the basis of capillary electrophoresis the immune privilege factor has been determined to be negatively charged. Capillary electrophoresis for IPF can be carried out using the Bio-Rad System with a CZE capillary (24 cm×25 μm), 0.1 M phosphate buffer, pH 2.5, at 6 kV.

Immune privilege factor can be manipulated at the protein level. Included within the scope of the present invention are IPF peptides which are differentially modified, e.g., by glycosylation, acetylation, phosphorylation, linkage to an antibody or other cellular ligand, etc. Any of numerous chemical modifications may be carried out by known techniques, including but not limited to specific chemical cleavage; acetylation, formylation, oxidation, reduction; metabolic synthesis in the presence of tunicamycin, etc.

5.2. Methods and Compositions for Use of and Administration of the Factor for Treating Inflammatory Diseases

The methods of the present invention comprise applying an effective amount of the central nervous system-derived immune privilege factor locally to a site to inhibit inflammation at the site. In a preferred embodiment the site is a site of nerve damage or unwanted inflammation in the central nervous system. Nerve damage or inflammation in the central nervous system may be due to a disease or disorder of the nervous system or due to a genetic disease or disorder of the nervous system including genetic degradative diseases. Such diseases or disorders, include but are not limited to nervous system injuries due to blunt trauma, disconnection of axons, a diminution or degeneration of neurons, autoimmune diseases or demyelination. Nervous system lesions which may be treated in a patient (including human and non-human mammalian patients) according to the invention include but are not limited to the following lesions of the central nervous system (including spinal cord, retina, brain) in which unwanted inflammation is present:

(i) traumatic lesions, including lesions caused by physical injury, blunt trauma, or associated with surgery, for example, lesions which sever a portion of the nervous system, or compression injuries;

(ii) ischemic lesions, in which a lack of oxygen in a portion of the nervous system results in neuronal injury or death, including cerebral infarction or ischemia, or spinal cord infarction or ischemia;

(iii) malignant lesions, in which a portion of the nervous system is destroyed or injured by malignant tissue which is either a nervous system associated malignancy or a malignancy derived from non-nervous system tissue;

(iv) infectious lesions, in which a portion of the nervous system is destroyed or injured as a result of infection, for example, by an abscess or associated with infection by human immunodeficiency virus, herpes zoster, or herpes simplex virus or with Lyme disease, tuberculosis, syphilis;

(v) degenerative lesions, in which a portion of the nervous system is destroyed or injured as a result of a degenerative process including but not limited to degeneration associated with Parkinson's disease, Alzheimer's disease, Huntington's chorea, multiple sclerosis, or amyotrophic lateral sclerosis;

(vi) lesions associated with nutritional diseases or disorders, in which a portion of the nervous system is destroyed or injured by a nutritional disorder or disorder of metabolism including but not limited to, vitamin B12 deficiency, folic acid deficiency, Wernicke disease, tobacco-alcohol amblyopia, Marchiafava-Bignami disease (primary degeneration of the corpus callosum), and alcoholic cerebellar degeneration;

(vii) neurological lesions associated with systemic diseases including but not limited to diabetes (diabetic neuropathy, Bell's palsy), systemic lupus erythematosus, carcinoma, or sarcoidosis;

(viii) lesions caused by toxic substances including alcohol, lead, or particular neurotoxins; and

(ix) demyelinated lesions in which a portion of the nervous system is destroyed or injured by a demyelinating disease including but not limited to multiple sclerosis, human immunodeficiency virus-associated myelopathy, transverse myelopathy or various etiologies, progressive multifocal leukoencephalopathy, and central pontine myelinolysis.

Other inflammatory diseases or conditions or disorders contributing to or caused by nerve injury for which the inhibitory factor of the present invention can be used to inhibit unwanted inflammation in the central nervous system and eye are, for example and not by way of limitation, AIDS-related dementia complex, HIV-related encephalopathy, post-polio syndrome, multiple sclerosis, myelitis, encephalitis, meningitis, rheumatic fever, complications and side-effects due to neurosurgery, subacute sclerosing panencephalitis, Huntington's disease, Devic's disease, Parkinson's disease, Alzheimer's disease, Sydenham chorea, posterior uveitis, anterior uveitis, sympathetic ophthalmia, retinitis, cystoid macular edema, optic neuritis, proliferative vitreoretinophathy, retinitis pigmentosa, glaucoma or a complication and/or side-effect from transplantation surgery or treatment of Parkinson's disease.

The present invention also provides methods for treatment by administration of a therapeutic composition comprising the immune privilege factor of the present invention and a pharmaceutically acceptable carrier to a subject to reduce inflammation at a selected local site. The subject is preferably an animal, including but not limited to animals such as cows, pigs, chickens, etc., and is preferably a mammal, and most preferably a human.

Various delivery systems are known and can be used to administer the immune privilege factor of the invention. The pharmaceutical compositions of the invention can be introduced into the central nervous system by any suitable route, including intraventricular and intrathecal injection, etc. Intraventricular injection may be facilitated by an intraventricular catheter, for example, attached to a reservoir, such as an Ommaya reservoir. The immune privilege factor may also be administered systemically by, for example, intravenous or intramuscular injection.

In a specific embodiment, the pharmaceutical compositions of the invention are administered locally to the area in need of treatment. This may be achieved by, for example, and not by way of limitation, local infusion during surgery, topical application, e.g., in conjunction with a wound dressing after surgery or directly onto the eye, by injection, by means of a catheter, or by means of an implant, said implant being of a porous, non-porous, or gelatinous material, including membranes, such as sialastic membranes, or fibers. In one embodiment, administration can be by direct injection at the site (or former site) of a malignant tumor or neoplastic or pre-neoplastic tissue. In another embodiment, the therapeutic composition can be administered to the eye by eye drops.

In yet another embodiment, the therapeutic composition can be delivered in a vesicle, in particular, a liposome see Langer, 1990, Science 249:1527-1533; Treat et al., 1989, in Liposomes in the Therapy of Infectious Disease and Cancer, Lopez-Berestein and Fidler (eds.), Liss, N.Y., pp. 353-365; Lopez-Berestein, ibid., pp. 317-327; see generally ibid.)

In yet another embodiment, the therapeutic composition can be delivered in a controlled release system. In one embodiment, a pump may be used (see Langer, supra; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used (see Medical Applications of controlled Release, Langer and Wise, 1974, (eds.), CRC Pres., Boca Raton, Fla.; Controlled Drug Bioavailability, Drug Product Design and Performance, Smolen and Ball (eds.), 1984, Wiley, N.Y.; Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled release system can be placed in proximity of the therapeutic target, i.e., the brain (see, e.g., Goodson, 1984, in Medical Applications of Controlled Release, supra, vol. 2, pp. 115-138).

Other controlled release systems are discussed in the review by Langer, 1990, Science 249:1527-1533.

The present invention also provides for pharmaceutical compositions comprising the immune privilege factor of the invention in a form which can be combined with or in combination with a pharmaceutically acceptable carrier, which compositions can be administered as described above. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The therapeutic composition, if desired, can also contain minor amounts of wetting or emulsifying agents, or pH buffering agents. These compositions can take the form of solutions, suspensions, emulsion, tablets, capsules, powders, sustained-release formulations and the like. The composition can be formulated with traditional binders and carriers such as triglycerides. Examples of suitable pharmaceutical carriers are described in “Remington's Pharmaceutical Sciences” by E. W. Martin. Such compositions contain a therapeutically effective amount of the therapeutic composition, together with a suitable amount of carrier so as to provide the form for proper administration to the patient. The formulation should suit the mode of administration.

In a preferred embodiment, the composition is formulated in accordance with routine procedures as a pharmaceutical composition adapted for local injection administration to human beings. Typically, compositions for local injection administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is administered by injection, an ampoule of sterile water for injection or saline can be provided so that the ingredients may be mixed prior to administration.

The therapeutic compositions of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The present invention also provides for the modification of the immune privilege factor such that it is more stable once administered to a patient, i.e., once administered it has a longer time period of effectiveness as compared to unmodified IPF. Such modifications are well know to those of skill in the art, e.g., polyethylene glycol derivatization (PEGylation), microencapsulation, etc. The amount of the therapeutic composition of the invention which is effective in the treatment of a particular disorder or condition will depend on the nature of the disorder or condition, and can be determined by standard clinical techniques. In general, the dosage ranges from about 0.01 mg/kg to about 10 mg/kg. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

The invention also provides a pharmaceutical pack or kit comprising one or more containers filled with one or more of the ingredients of the pharmaceutical compositions of the invention.

The following series of examples are presented by way of illustration and not by way of limitation on the scope of the invention.

### 6. EXAMPLE

### Migration of Monocytes (Macrophages) to Optic Nerve or Sciatic Nerve

6.1. Migration of Macrophages to Optic Nerve or Sciatic Nerve Segments

In order to evaluate the ability of optic nerve or sciatic nerve segments to induce migration of macrophages, modified Boyden chambers were used. One chamber contained a blood leukocyte population containing monocytes. The leukocyte population was collected from rat blood by standard density centrifugation on a Percoll gradient (1.077 g/ml). Sprague-Dawley (SPD) rats 12-14 weeks of age were over-anesthetized with chloroform and 7 ml of blood were collected from the heart into a heparinized 10 ml syringe with an 18 gauge needle. The blood was diluted 1:1 in cold phosphate buffered saline (PBS) in a hepatinized tube and after five minutes layered on to the Percoll gradient. The gradient was centrifuged at 400×g for 25 minutes at 20° C. The buffy coat was removed and washed slowly twice with Dulbecco's Modified Eagle Medium (DMEM) culture medium to remove the platelets. The cells were counted and suspended at 10,000/ml. The cells, i.e., the leukocyte population containing monocytes, i.e., macrophages, were used as soon as possible to avoid adherence.

The other chamber contained segments of either optic nerve or sciatic nerve. The nerve segments were isolated from Sprague-Dawley 12-14 week old rats. The rats were over-anesthetized as above and optic and sciatic nerves were removed aseptically and placed in cold PBS. The nerves were cleaned of debris and cut into 1 mm segments. The segments were placed into the chamber containing 200 μl DMEM. A Sartorius filter which is impermeable to cells was placed over the top of the nerve-containing chamber, carefully avoiding the introduction of air under the filter. The chamber was closed and 500 μl of the DMEM-leukocyte solution was added. The chambers were incubated and stopped at 1, 3 or 24 hours by opening the chamber and placing the filter in 70% ethanol for 5 minutes.

Macrophages which are induced to migrate will contact the filter and adhere thereto and are subsequently visualized by microscopy. Briefly, after the filter has been fixed in 70% ethanol for 5 minutes, it is transferred to ddH₂O for one minute, placed in hematoxylin solution (Sigma Chemical Co.) for one minute, placed in ddH₂O for one minute and then placed in tap water for three minutes. The filter was dried by placing it in 70% ethanol for 2 minutes, 100% ethanol for two minutes and in 80% ethanol/20% butanol for 5 minutes. The filter was clarified by placing it in xylene for 4 minutes. The filter was then placed in 70% ethanol for two minutes and mounted on a slide with glycerin. The cells on the filter were counted using a Nikon inverted fluorescence microscope. Images of the cells were captured and digitized with an Applitek CCD camera and a Scion LG-3 framegrabber board using a Macintosh Quadra 840 AV. Analysis was performed using NIH-Image V. 1.55 by Wayne Rasband.

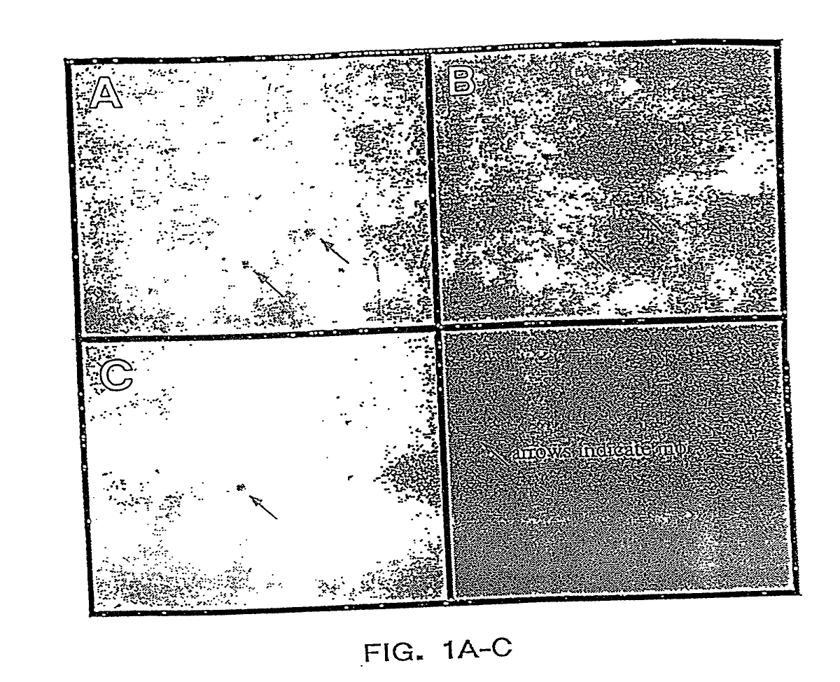

FIGS. 1A-C show the results of a typical experiment as described above. FIGS. 1A-C are fluorescence micrographs showing the relative migratory response of the macrophages to either optic nerve (FIG. 1A), sciatic nerve (FIG. 1B) or to control medium (FIG. 1C). It is apparent that more macrophages were induced to migrate towards sciatic nerves than towards optic nerves or control medium.

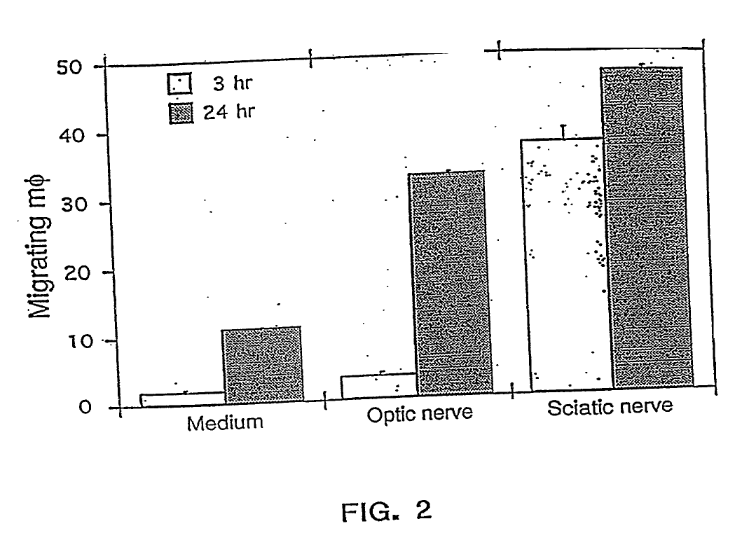

When the kinetics of macrophage migration was determined more macrophages were induced to migrate towards sciatic nerve than optic nerve; however, over longer incubation periods the difference in the number of macrophages migrating to sciatic nerve as compared to optic nerve decreased. This is shown in FIG. 2, where an incubation period of 3 hours (open bars) is compared to 24 hours (solid black bars). After both incubation periods the number of macrophages migrating towards sciatic nerves was significantly higher than those migrating towards optic nerves, however, the difference was far greater after the shorter 3 hour incubation.

As a control, sciatic and optic nerves were incubated alone to determine whether macrophages associated with the nerves at the time of excision contributed to the macrophage population adhering to the filters. In each of these control experiments no macrophages were found on the filter. Therefore, the macrophages adhering to the filter were only those external to the nerve tissue itself. Hence, these results indicate that fewer macrophages are induced to migrate towards optic nerve than towards sciatic nerve and that the attraction is slower.

6.2. Migration of Macrophages to Medium Conditioned by Optic Nerve or Sciatic Nerve Segment

In another series of experiments, it was determined that the factor which induced the migration of the macrophages is a soluble factor released by nerve tissue of the central nervous system. This tissue is mainly composed of non-neuronal cells which envelop the axons of the nerve segments used in the experiments, i.e., various glial cells in the optic nerve tissue and Schwann cells in the sciatic nerve tissue. It should be noted that the optic nerve and sciatic nerve segments do not contain nerve cell bodies but only axons surrounded by non-neuronal cells. Accordingly, factors released by these segments into the medium are most likely to be from the non-neuronal cells.

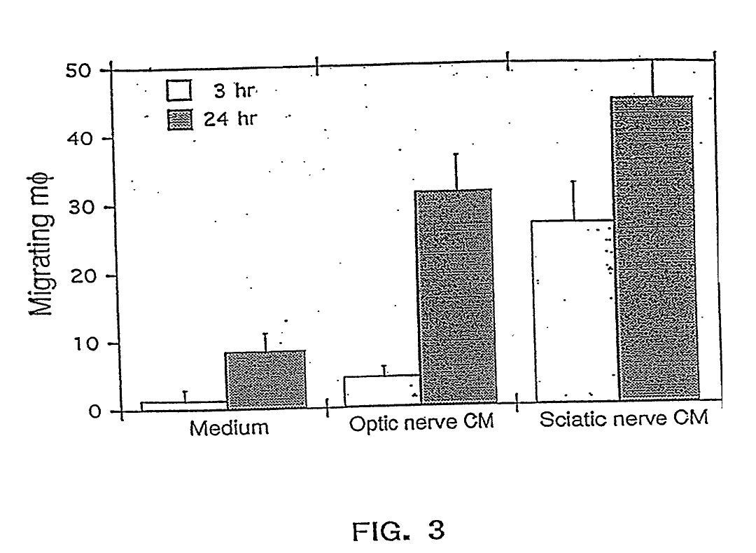

Optic and sciatic nerves were collected as described in Section 6.1 and placed separately in 1 ml DMEM in a 24 well tissue culture plate. The plates were incubated for 24 hours at 5t CO₂, 75% relative humidity, 37° C. The medium was collected for each nerve type and pooled (4 ml from 4 nerves, either optic or sciatic) and filtered through a 0.22 micron filter. The conditioned medium was stored at −70° C. until used. The prepared sciatic nerve conditioned medium (SNCM) and the optic nerve conditioned medium (ONCM) were placed in modified Boyden chambers as described above in Section 6.1 and the relative effect each conditioned medium had on macrophage migration was determined. Representative results of these experiments are shown in FIG. 3. These results are very similar to those using actual nerve tissue rather than nerve conditioned medium.

Hence, these results indicate that a soluble factor exists, that is released from nervous tissue and is capable of inducing the migration of macrophages towards it. This chemoattractant factor is present in both sciatic and optic nerve tissue but its effect appears to be delayed or inhibited by some other factor present in or released by the optic nerve tissue.

6.3. Dilution Curve of Sciatic and Optic Nerve Conditioned Medium

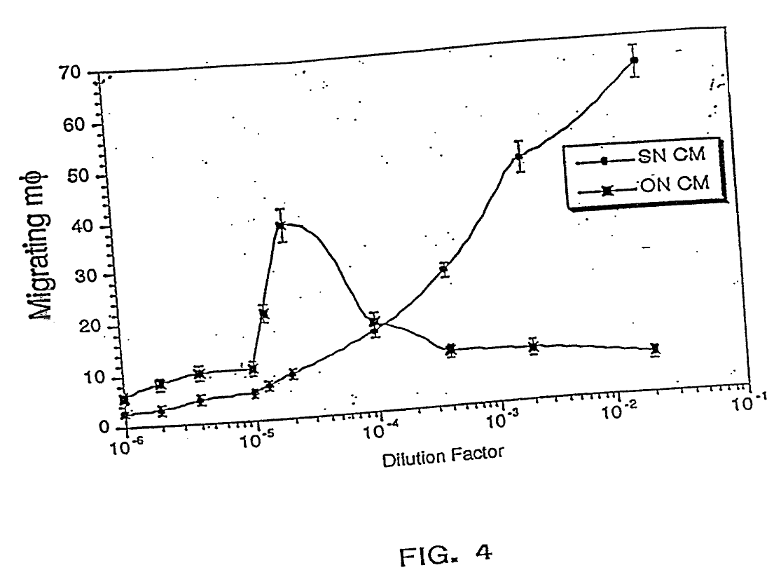

Dilution studies of optic nerve or sciatic nerve conditioned medium (ONCM and SNCM, respectively) were carried out to determine the concentration of chemoattractant activity associated with the nervous tissue. Various dilutions of ONCM and SNCM were incubated with macrophages in Boyden chambers as described above in Section 6.1, and the results are depicted in FIG. 4.

FIG. 4 presents a graph of the amount of macrophages migrating towards SNCM versus the concentration of SNCM in units of relative dilution (closed circles); and of the amount of macrophages migrating towards ONCM versus the concentration of ONCM in units of relative dilution (closed squares). FIG. 4 shows that SNCM has half-maximal chemoattractant activity at a dilution of 1:500 while ONCM has chemoattractant activity in the 1:20,000 to 1:100,000 range and has no activity higher or lower than that concentration range. The dilution curve pattern suggests the presence of both a chemoattractant and an inhibitor, with the inhibitor diluting out before the chemoattractant. The presence of an inhibitor was confirmed in a mixing experiment in which the addition of ONCM to SNCM caused a reduction in macrophage migration upwards of 80%, see Section 7, infra.

6.4. Association of Macrophage Morphology and Migratory Activity

In another series of experiments, the morphology of the macrophages induced to migrate by optic or sciatic nerve conditioned medium was studied. It was observed that in addition to the difference in migratory response between ONCM and SNCM, the macrophages have different morphologies when incubated in the different conditioned media. A monocyte cell line, 14M1, was used for the morphology studies. The cell line is a transformed bone marrow stem cell that differentiates into macrophage-like cells (Zipori et al., 1984, J. Cell Physiol. 118:148-152). 14M1 cells are CSF-1 dependent and behave like stem cells by differentiating into a macrophage-like cells when stimulated with lipopolysaccharide (LPS) or latex beads. 14M1 cells and blood monocytes were plated in 24 well plates (5000 cells/ml/well). Either pieces of nerve (optic nerve of sciatic nerve) or 0.5 ml of medium conditioned by these nerves were added to the wells. Plates were imaged at 0, 24, 48, 72 and 96 hours.

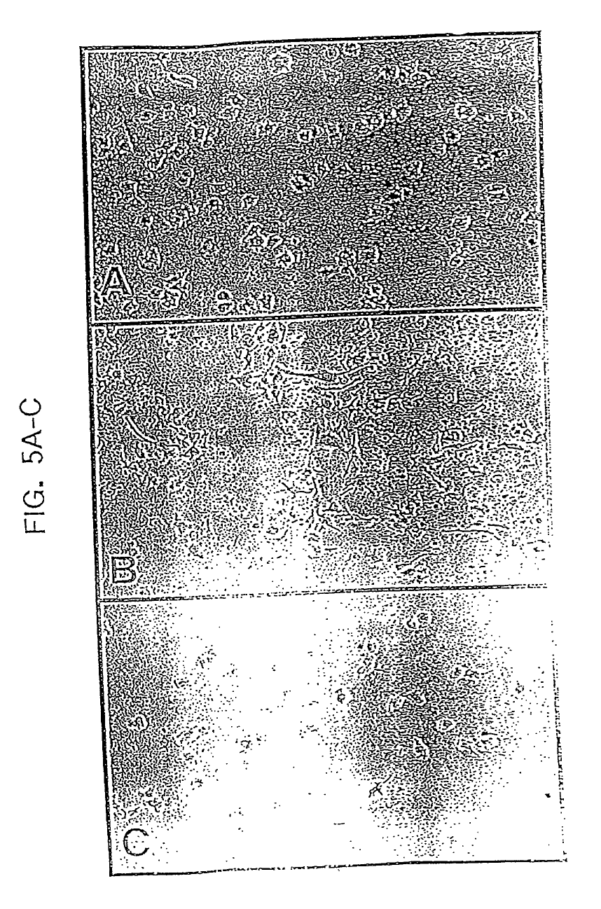

Results are presented in FIG. 5A-C. FIG. 5A shows 14M1 cells incubated in ONCE; FIG. 5B shows 14M1 cells incubated in SNCM; FIG. 5C shows 14M1 cells incubated in control medium. Monocytes incubated with optic nerve tissue or ONCM had few processes and a more radial cytoplasm while monocytes cells incubated with sciatic nerve tissue or SNCM had more processes and a much more polar cytoplasm (spindle shape). The cells were scored based on their morphology in the different incubatory environments. The number of migratory macrophages, i.e., spindle shape morphology, was greater in cells exposed to the sciatic nerve tissue or SNCM incubation conditions. The difference in the number of migratory-type cells was most pronounced in the period between 24 and 72 hours after the start of the incubation.

### 7. EXAMPLE

### CNS Macrophage Migration Inhibitory Factor (IPF) Associated with Optic Nerve Tissue or Optic Nerve Conditioned Medium

The dilution curve of ONCM, see FIG. 4, demonstrates that ONCM has different effects on macrophage migration at different dilutions; at lower concentrations it is a chemoattractant for macrophages and at higher concentrations it has no chemoattractant effect or may even be inhibitory. In order to determine whether ONCM at higher concentrations does have an inhibitory effect on macrophage migration, experiments were carried out as above using the Boyden chambers in which ONCE was added to SNCM and the mixture was tested for its effect on macrophage migration.

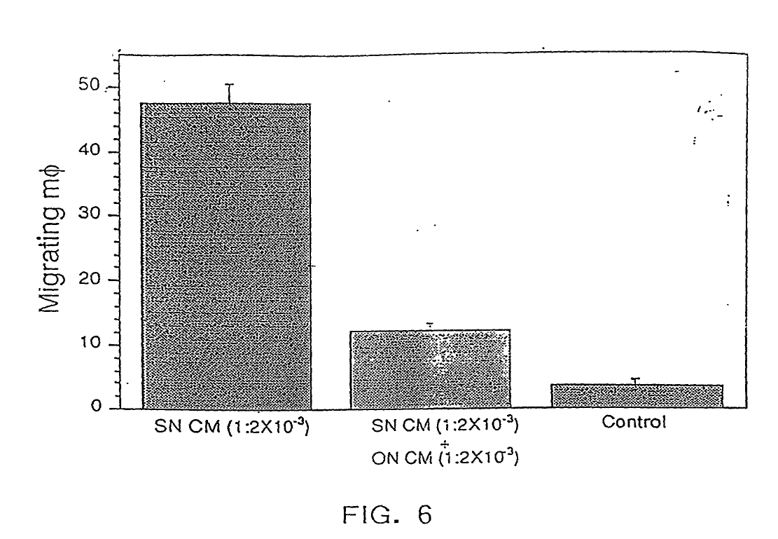

A comparison was made between SNCM at a dilution of 1:2000 (a level wherein there is near maximal effect on inducing macrophage migration, see FIG. 4) and a mixture of equal parts SNCM (1:2000 dilution) and ONCM (1:2000 dilution, which has no effect on macrophage migration, see FIG. 4). Standard DMEM culture medium was used as a control. The results are depicted in FIG. 6. It is apparent that SNCM alone induced macrophage migration but, by comparison, SNCM mixed with ONCM induced very few macrophages to migrate. It is concluded that there is a factor in ONCE which inhibits macrophage migration induced by SNCM by upwards of 80%.

### 8. EXAMPLE

### Co-Elution of Macrophage Migration Inhibitory Factor from Brain and Optic Nerve

Optic nerve- and brain-conditioned media were prepared by incubating optic nerve or sliced brain tissue freshly excised from rats in saline and placed in an incubator (5% CO₂, 75% relative humidity, 37° C.). After 1 hour, the conditioned media were centrifuged in order to remove cellular debris, the supernatants collected and total protein was determined by the Bradford Assay.

Medium conditioned by optic nerve (ONCM) containing a total of 1.8 mg protein or by brain (BCM) containing a total of 5 mg protein was subjected to size exclusion chromatography on a “SUPERDEX”™ 75 column (Pharmacia, Uppsala, Sweden). The collected fractions were tested for the ability to inhibit macrophage migration as described in Section 6.1, supra.

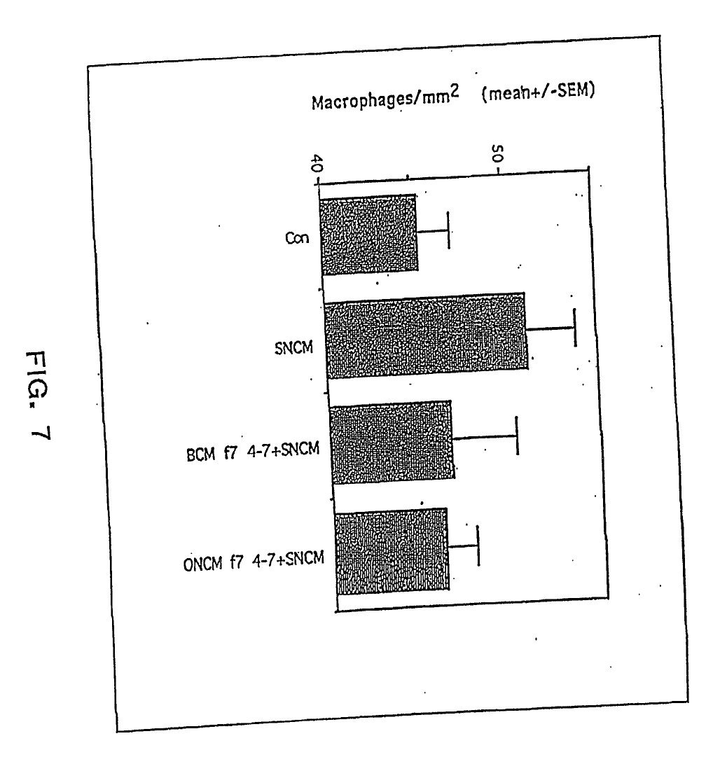

The collected fractions containing the activity were further subjected to reverse phase high pressure liquid chromatography (HPLC). The fractions were run over a C-18 column with 5 mm pores. The gradient was run with 0-30% acetonitrile in double distilled water for 30 minutes with a flow rate of 0.8 ml per minute; each collected fraction contained 0.6 ml. The collected fractions were tested as described above and the activity derived from both sources again co-eluted at 4-7 minutes. Results are presented in FIG. 7. The control (Con) was DS4EM tissue culture medium.

FIG. 7 clearly demonstrates that the macrophage inhibitory activity derived from optic nerve or brain tissue elutes in the same fractions collected from the HPLC column, indicating that it is the same molecule.

### 9. EXAMPLE

### Inhibition of Macrophage Phagocytic Activity by Optic Nerve Conditioned Medium

Sprague-Dawley (SPD) rats, aged 10-12 weeks, were overanesthetized with chloroform and 10 ml blood was withdrawn from the heart into a heparinized 10 ml syringe with a 21 gauge needle. Macrophages were collected by density centrifugation on a Percoll gradient (1.077 g/ml). The blood was diluted with phosphate buffered saline (PBS) at room temperature and after 5 minutes layered onto the Percoll solution. The cells were centrifuged at 400×g for 25 minutes at 25° C. and the buffy coat was removed, the cells were washed twice with Dulbecco's modified Eagle's medium (DMEM), resuspended at 1×10⁶ macrophages per ml and placed in an incubator (5% CO₂, 75% relative humidity, 37° C.).

Optic nerve and sciatic nerve conditioned medium was prepared by incubating optic nerve or sciatic nerve freshly excised, from the same rats from which blood was withdrawn, in saline and placed in an incubator (5% CO₂, 75% relative humidity, 37° C.). After 1 hour, the conditioned media was centrifuged in order to remove cellular debris, the supernatants collected and total protein was determined by the Bradford Assay.

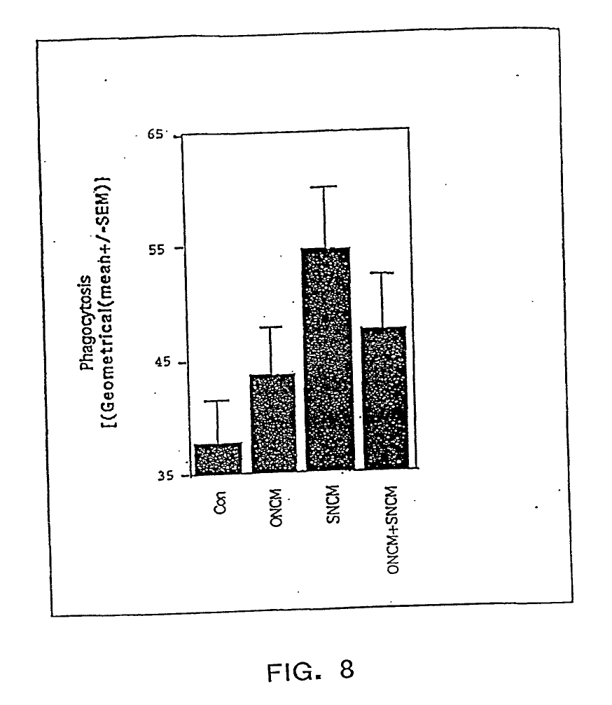

The isolated macrophages were diluted to a concentration of 80,000 cells per ml DMEM and placed in Teflon bags in a total volume of 5 ml (Becton Dickinson, Franklin Lakes, N.J.). Conditioned medium (15 mg protein total) derived from optic nerve (ONCM) or sciatic nerve (SNCM) or a mixture of the two was added to the cells. Phagocytosis was determined by the addition of FITC fluorescent beads (Polyscience, Warrington, Pa.) for 12 hours and subsequent recording by fluorescence absorbency cytometry (FACSCAN), using the CellQuest software (Becton Dickinson, Franklin Lakes, N.J.). Results were expressed as mean percentages (±SEM) of FITC staining. Macrophages without conditioned medium added were used as a control. Results are presented in FIG. 8. Data are expressed as the geometric mean of triplicate experiments. FIG. 8 clearly shows that ONCM containing the immune privilege factor inhibits macrophage phagocytic activity as compared to SNCM.

### 10. EXAMPLE

### Characterization of the Immune Privilage Factor

10.1. Heat Resistance

The CNS derived immune privilege factor was tested for heat stability. Optic nerve conditioned medium (ONCM) and sciatic nerve conditioned medium (SNCM) was prepared as described above in Section 6 and tested in a modified macrophage migration assay as described below.

The macrophage migration assay was modified so that the macrophages were first induced to chemotact by N-formyl-Met-Leu-Phe (N-f-MLP), (Dureus et al., 1993, Cell Mol. Neurobiol. 13:541-546) a known macrophage chemoattractant, in order to more easily determine the ability of the conditioned medium to inhibit macrophage migration. Briefly, the assay was carried out as follows. The conditioned media were placed in the bottom half of a Boyden chamber containing 200 μl DMEM containing 40 μg/μl N-f-MLP. A Sartorius filter with 8 μm pores was placed on top of the DMEK/N-f-MLP solution. Macrophages were isolated as described above and were stained with 10.7 μM Hoechst 34422 vital nuclear stain for 10 minutes at 37° C. and washed twice with PBS. The macrophages were added to the upper chamber of the Boyden chamber. The migration assay was stopped after 16 hours and the macrophages adhering to the filter were visualized as described above.

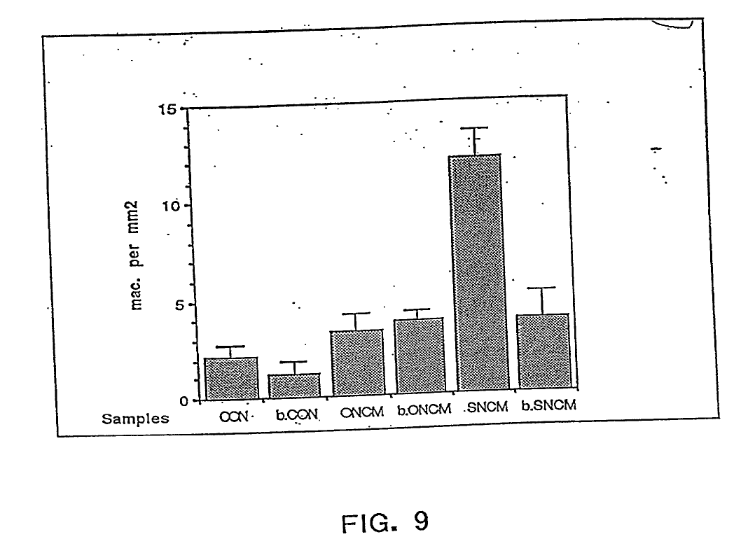

Native and boiled (100° C. for 10 minutes) samples of ONCM at 0.5 mg/ml total protein with 10⁻⁷ M K-f-MLP, and native and boiled samples of SNCM at 0.5 mg/ml total protein were added to macrophages as described above. Results are presented in FIG. 9. Native (con) and boiled DMEM (bcon) were used as controls.

FIG. 9 shows that the boiling of ONCM does not affect the ability of the immune privilege factor to inhibit macrophage migration. The activity in SNCM that induces macrophage migration is not heat stable.

10.2. Protease Sensitivity

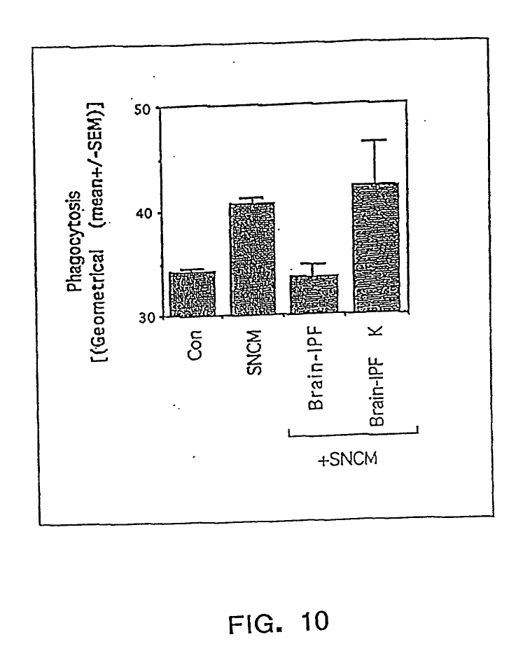

Brain tissue conditioned medium (BCM) containing the immune privilege factor was prepared as described above and BCM at 500 mg total protein was treated with 40 mg Proteinase K (Merck, Rahway, N.J.) for 45 minutes. The samples were then boiled for 30 minutes to denature and inactivate the Proteinase K. The BCM samples were then tested for their ability to inhibit macrophage phagocytosis as described above in Section 9. Results are shown in FIG. 10. Sciatic nerve conditioned medium (SNCM) and DMEM alone were used as controls.

FIG. 10 shows that the immune privilege factor is sensitive to protease treatment indicating that the factor is a peptide.

### 11. EXAMPLE

### Purification and Identification of the Factor from Optic Nerve

Optic nerves were excised from adult Sprague-Dawley rats as described in Section 6.1 and were incubated for one hour in PBS to yield optic nerve conditioned medium (ONCM). The ONCM was subjected to gel filtration chromatography using a SUPEROSE™ 12 column (a gel filtration medium, Pharmacia, Uppsala, Sweden). The flow rate through the column was 0.5 ml/minute, the running buffer was PBS diluted 1:3 in distilled water and the fractions were collected as 2.5 ml aliquots. Every two consecutive fractions were combined and analyzed using the macrophage migration assay with modifications described in Section 10.1.

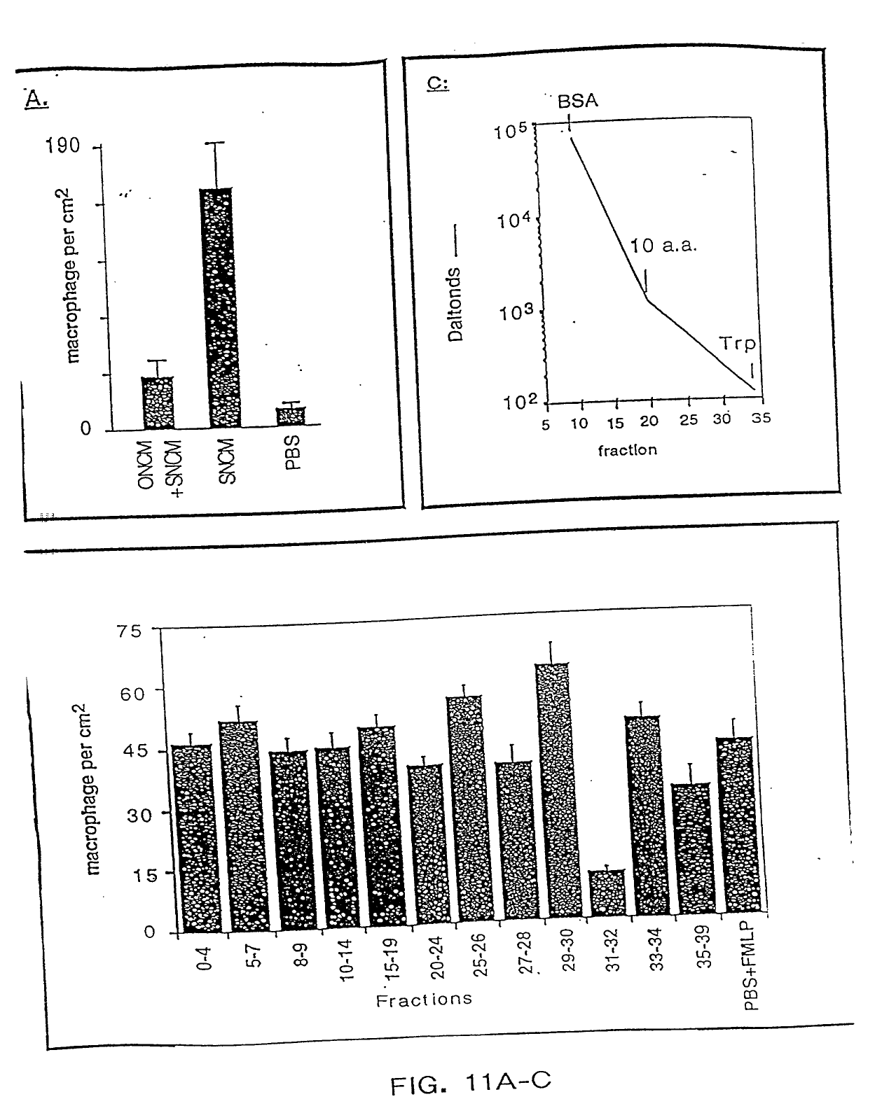

A control experiment was conducted in which the inhibitory effect of unfractionated ONCM on the migration of macrophages induced by SNCM was determined. The unfractionated ONCM was the starting material which was fractionated over the gel filtration column. The experiment was carried out under the same conditions using the same dilutions as in FIG. 6 except that 4 times more macrophages were used. The results are depicted in FIGS. 11A-C.

FIG. 11A shows the result of the control experiment. This result is essentially the same as depicted in FIG. 6 and demonstrates that the prepared unfractionated ONCM had the same inhibitory effect on macrophage migration as the previously prepared ONCM and that the use of four times more macrophages did not influence the overall result. The data in FIG. 11A represents the mean ±SEM (mean of 6 visualized fields from each filter in duplicate; n=12).

FIG. 11B shows the inhibitory effect of the various fractions on macrophage migration induced by N-f-MLP. The data in FIG. 11B represents the mean ±SEM (mean of 6 visualized fields from each filter in duplicate; n=12). Fractions 31 and 32 from the SUPEROSE™ 12 column caused a decrease of approximately 300% in the capacity of macrophages to respond to N-f-MLP as compared to the other fractions and the PBS/N-f-MLP control sample, indicating that fractions 31 and 32 contain essentially all of the active immune privilege factor.

In order to determine the approximate size of the immune privilege factor present in ONCM, molecular weight markers were subjected to gel filtration chromatography under the same conditions. Markers used were Bovine Serum Albumin (BSA), a 10 amino acid peptide and the amino acid tryptophan. FIG. 11C depicts the elution profile of the various markers of known molecular weight, i.e., standard curve. BSA with a molecular weight of 65,000 Daltons eluted in fractions 11 and 12, the 10 amino acid peptide with a molecular weight of about 1500 Daltons eluted in fractions 21 and 22 and tryptophan with a molecular weight of about 200 Daltons eluted in fraction 34. By extrapolation of the standard curve plotted for molecular weight, the immune privilege factor, eluting in fractions 31 and 32, has a molecular weight of approximately 350 Daltons.

The immune privilege factor, after pre-column derivatization with orthophtal aldehyde (OPA) and 9-fluoro-enile methylchloroformate (SM/OK) and elution of the derivative from a C-18 reverse phase column, was subjected to amino acid analysis using a Hewlett Packard 1096 Amino Acid Analyzer. The amino acid composition of the immune privilege factor was determined to be glutamic acid, serine and glycine.

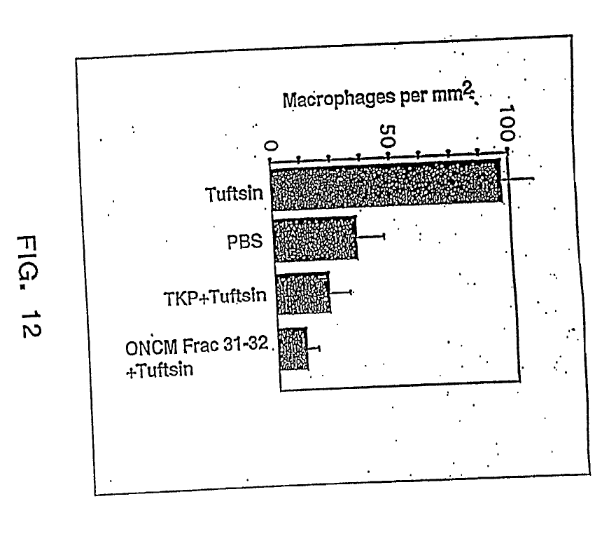

In another experiment, the macrophage migration inhibitory activity of the immune privilege factor of the present invention was compared to that of a known macrophage chemoattractant, tuftsin, and that of a known macrophage inhibitor, the tri-peptide Threonine-Lysine-Proline (TKP). The macrophage migration assays were carried out as described above in Section 6.1. Results are presented in FIG. 12.

FIG. 12 shows that the immune privilege factor of the present invention has similar activity in blocking the effect of tuftsin as it has in blocking the effect of N-f-MLP. In addition, it has a similar inhibitory activity as TKP.

### 12. EXAMPLE

### Purification of the Factor from Brain Tissue

Brain tissue was excised from adult Sprague-Dawley rats as described in Section 9 and was incubated for one hour in saline to yield brain tissue conditioned medium (BCM). Optic nerve was excised from adult Sprague-Dawley rats as described and was incubated for one hour in saline to yield optic nerve conditioned medium (ONCM). ONCM and BCM containing a total of 250 μg of protein were subjected to gel filtration chromatography using a SUPEROSE™ 12 column (a gel filtration medium, Pharmacia, Uppsala, Sweden). The flow rate through the column was 0.5 ml/minute, the running buffer was PBS diluted 1:3 in distilled water and the fractions were collected as 2.5 ml aliquots. Every two consecutive fractions were combined and analyzed using the macrophage migration assay with modifications described in Section 10.1.

The collected fractions containing the immune privilege factor as measured by inhibition of macrophage migration were combined and were subjected to reverse phase high pressure liquid chromatography (HPLC). The fractions were run over a C-18 column with 5 mm pores. The gradient was run with 0-30% acetonitrile in double distilled water for 30 minutes with a flow rate of 0.8 ml per minute, each collected fraction contained 0.6 ml. The collected fractions were then tested for macrophage migration inhibitory activity. Following separation on HPLC, the factions containing the immune privilege factor were combined and subjected to separation on thin layer chromatography (TLC) on a silica gel 60 precoated plastic foil plate (Merck, Rahway, N.J.) using butanol:acetic acid:water (4:4:1) as the separation buffer. The active band was excised and extracted from the silica gel into 100 μl double distilled water. Assuming a 90% loss during purification and starting with 250 μg total protein, the purified IPF represents approximately an equivalent amount of IPF derived from 25 μg total protein of the original material. Since the extracted IPF is in a total volume of 100 μl, every microliter of purified IPF represents the equivalent amount of IPF derived from 0.25 μg total protein.

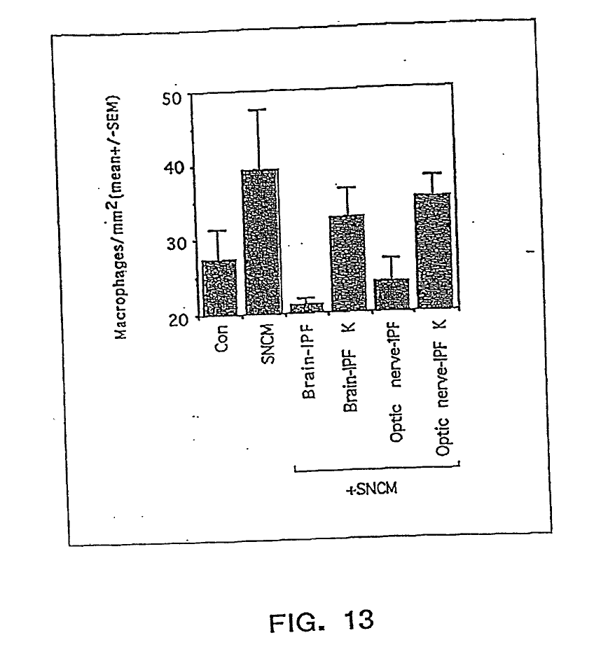

The extracted IPF in this example was tested at a dilution of 5×10⁻⁴ relative to the original optic nerve and brain tissue conditioned media for sensitivity to Proteinase K as measured by inhibition of macrophage migratory activity. Proteinase K treatment was for 30 minutes at 37° C. using 80 mg Proteinase K per 100 ml of Tris HCl buffer, pH 7.5, followed by 15 minutes at 100° C. to inactivate the protease. FIG. 13 shows the ability of the purified factor derived from both brain and optic nerve to inhibit macrophage migration and its sensitivity to protease treatment.

### 13. EXAMPLE

### Purification of the Factor from Brain Tissue by Ion-Exchange

Brain tissue was excised from adult Sprague-Dawley rats as described in Section 9 and was incubated for one hour in saline to yield brain tissue conditioned medium (BCM). BCM containing a total of 250 μg of protein was subjected to gel filtration chromatography using a SUPEROSE™ 12 column (a gel filtration medium, Pharmacia, Uppsala, Sweden). The flow rate through the column was 0.5 ml/minute, the running buffer was PBS diluted 1:3 in distilled water and the fractions were collected as 2.5 ml aliquots. Every two consecutive fractions were combined and analyzed using the macrophage migration assay with modifications described in Section 10.1.

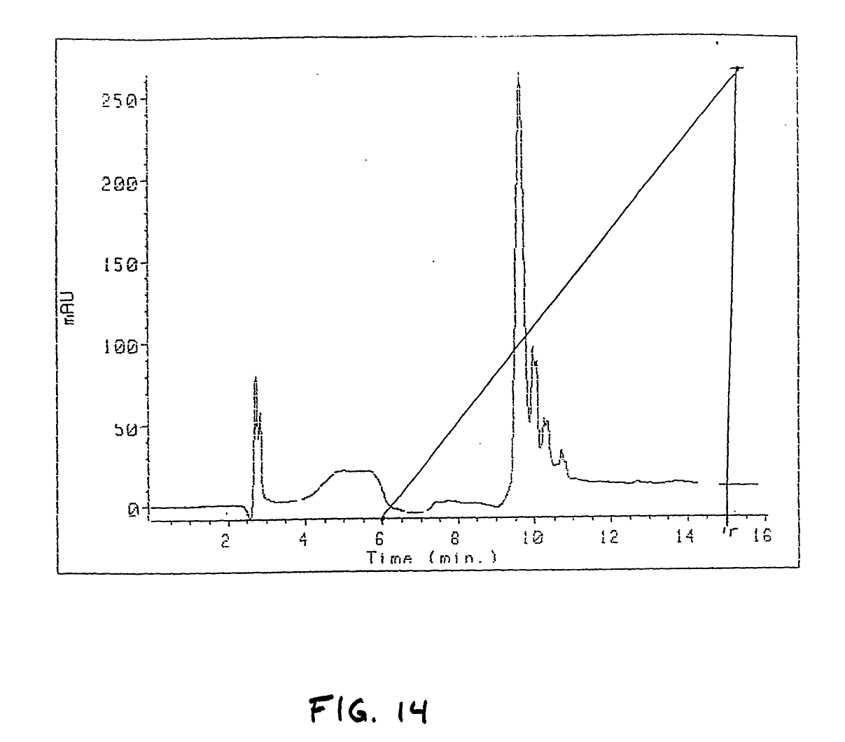

The collected fractions containing the immune privilege factor as measured by inhibition of macrophage migration were combined and were subjected to reverse phase high pressure liquid chromatography (HPLC). The fractions were run over a C-18 column with 5 mm pores. The gradient was run with 0-30% acetonitrile in double distilled water for 30 minutes with a flow rate of 0.8 ml per minute, each collected fraction contained 0.6 ml. The collected fractions were then tested for macrophage migration inhibitory activity. Following separation on HPLC, the factions containing the immune privilege factor were combined and subjected to ion exchange column chromatography with a PolyWAX (200×4.6) column (PolyLC, Inc., Maryland, USA) with a flow rate of 1 ml/min of Buffer A (20 mM Tris, pH 8) for 0 to 6 minutes and at a gradient of 0-100% (6 to 15 minutes) Buffer B (20 mM Tris, pH 8, 250 mM NaCl). The elution profile is shown in FIG. 14.

FIG. 14 shows that IPF can be purified by ion exchange column chromatography and elutes off the column at 10 minutes at/with approximately 100 mM NaCl.

### 14. EXAMPLE

### Inhibition of Cell Adhesion by Immune Privilege Factor

14.1. Inhibition of Macrophage Adhesion

Blood was obtained from healthy human volunteers and macrophages were isolated by density centrifugation on a Percoll gradient (1.077 g/ml). The blood was diluted with phosphate buffeted saline (PBS) at room temperature and after 5 minutes layered onto the Percoll solution. The cells were centrifuged at 400×g for 25 minutes at 25° C. and the buffy coat was removed, the cells were washed twice with Dulbecco's modified Eagle's medium (DMEM), resuspended at 1×10⁶ macrophages per ml and placed in an incubator (5% CO₂, 75% relative humidity, 37° C.).

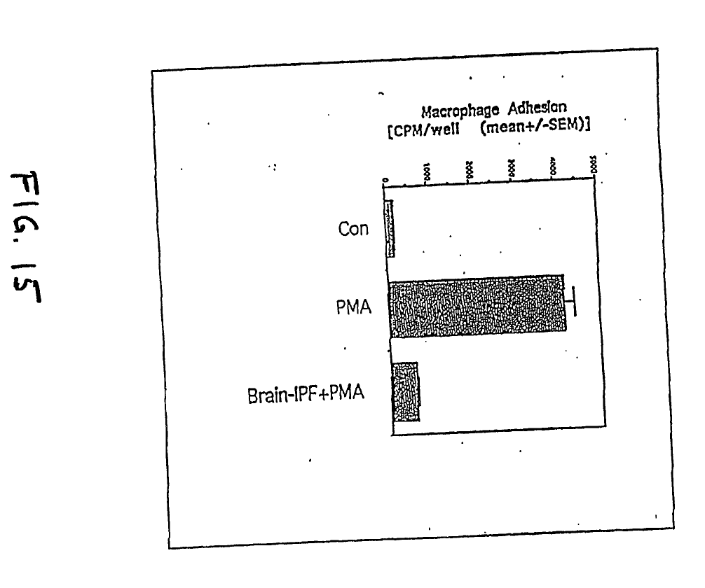

The macrophages were labeled with chromium⁵¹ and added to 96 well plates precoated with fibronectin or retinal extracellular matrix in RPMI 1640 medium supplemented with 2% bovine serum albumin, 1 mM Ca²⁺, 1 mM Mg²⁺ 1% sodium pyruvate, 1% glucose and 1% HEPES buffer pH 7.0-7.4 (adhesion medium) at 10⁵ cells per 100 ml adhesion medium. The labeled macrophages were preincubated with IPF purified as described in Section 12, supra, at a 1:100 dilution relative to the conditioned medium for 60 minutes at 37° C. The macrophages were then activated with 25 ng/well phorbol 12-myristate-13-acetate (PMA) (Sigma Chemical Co., St. Louis Mo.). The wells were then washed 3 times to remove non-adherent cells. Radiolabeled adherent macrophages were examined through an optical microscope to ensure cell viability and adequate washings. The cells were then lysed and the supernatants collected for gamma counting. Results are presented in FIG. 15 and are expressed as mean (±SEM) counts per minute (cpm) in quadruplicate wells from each experimental group.

The results shown in FIG. 15 are from the 96 well plates coated with extracellular matrix. Similar results are seen when the plates are coated with fibronectin. PMA-free buffer served as a control. Adherence in the presence of PMA only is designated by PMA. The ability of immune privilege factor derived from rat to inhibit human macrophage adhesion shows that the factor has cross species reactivity. It further shows that IPF effects not only macrophages but other immune cells as well, indicating a broad reactivity towards immune cells in general.

14.2. Inhibition of T Cell Adhesion

Blood was obtained from healthy human volunteers and T cells were isolated by diluting the blood 1:1 with PBS and then centrifuging the dilutant for 20 minutes at 700×g (1600 rpm) to collect the mononuclear interphase. The monocytes were then excluded by filtering the interphase through nylon wool tubes (Uni-Sorb tubes, NovaMed, Israel). The purified T cells were centrifuged again for 15 minutes at 350×g (800 rpm). The pellet was resuspended in RPMI medium at 10⁶ cells per ml.

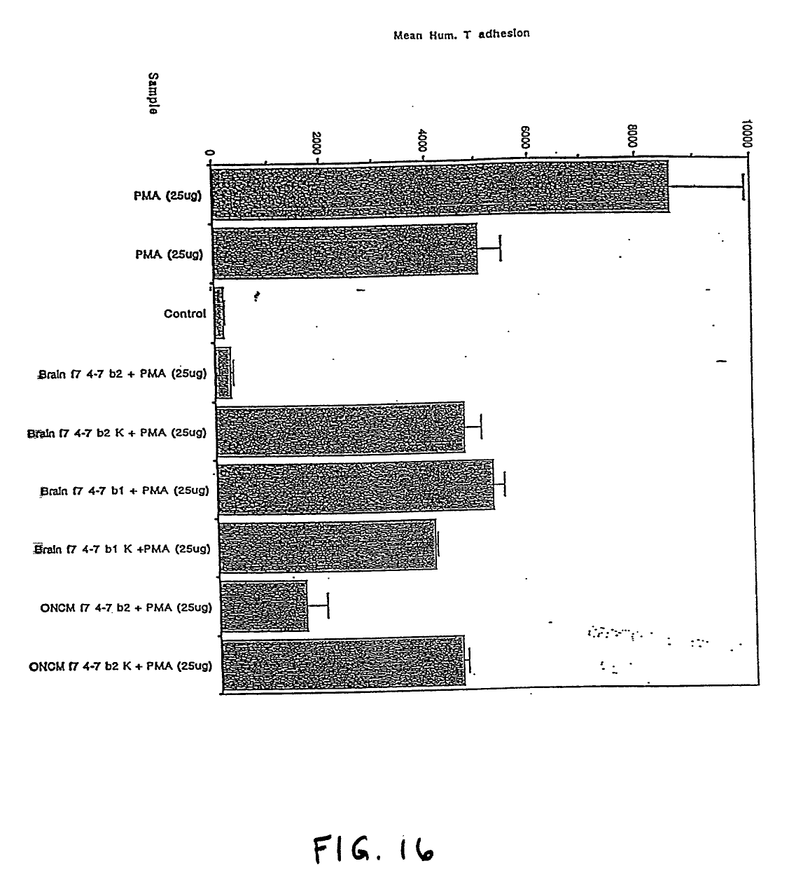

The isolated T cells were labeled with chromium⁵¹ and added to 96 well plates precoated with fibronectin or retinal extracellular matrix in RPMI 1640 medium supplemented with 2% bovine serum albumin, 1 mM Ca²⁺, 1 mM Mg²⁺ 1% sodium pyruvate, 1% glucose and 1% HEPES buffer pH 7.0-7.4 (adhesion medium) at 10⁵ cells per 100 ml adhesion medium. The labeled T cells were preincubated with 2 different batches of brain-derived immune privilege factor as described in Section 13.1, supra, or optic nerve conditioned medium with or without Proteinase K treatment (10 μg) for 60 minutes at 37° C. After incubation the T cells were activated with 25 ng/well PMA. The wells were then washed 3 times to remove non-adherent cells. Radiolabeled adherent T cells were examined through an optical microscope to ensure cell viability and adequate washings. The cells were then lysed and the supernatants collected for gamma counting. Results are demonstrated in FIG. 16 and are expressed as mean (±SEM) counts per minute (cpm) in quadruplicate wells from each experimental group.

The results shown in FIG. 16 are from the 96 well plates coated with extracellular matrix. Similar results are seen when the plates are coated with fibronectin. PMA-free buffer served as a control. Adherence in the presence of PMA only is designated by PMA. FIG. 16 shows that the immune privilege factor inhibits adhesion of PMA activated human T cells to ECM or fibronectin. The ability of immune privilege factor derived from rat to inhibit human T cell adhesion shows that the factor has cross species reactivity. This ability was destroyed upon protease treatment.

Similar inhibition of adhesion of human T cells to ECM and fibronectin were seen when the T cells were activated with macrophage inflammatory protein 1β (MIP-1β). For a discussion of MIP-1β, see, e.g., Fahey et al., 1992, J. Immunol. 148:2764; Taub et al., 1993, Science 260:355; and Tanaka et al., 1993, Nature 361:79.

The ability of immune privilege factor derived from rat to inhibit human T cell adhesion also shows that IPF affects T cells as well as macrophages which indicates a broad reactivity of IPF towards immune system cells in general.

### 15. EXAMPLE

### Upregulation of the FAS Receptor

Blood was obtained from healthy human volunteers and T cells were isolated by the method described in Section 13.2, above.

The isolated T cells were incubated at 5% CO₂, 37° C., and 75% relative humidity for 17 hours in 15 ml polypropylene tubes in RPMI medium (1.5 ml, 10⁶ cells/tube) in the presence of sciatic nerve conditioned medium (SNCM; 200 μg total protein/tube) or 10 μl immune privilege factor purified as described in Section 12, supra, or both. RPMI was added alone as a control.

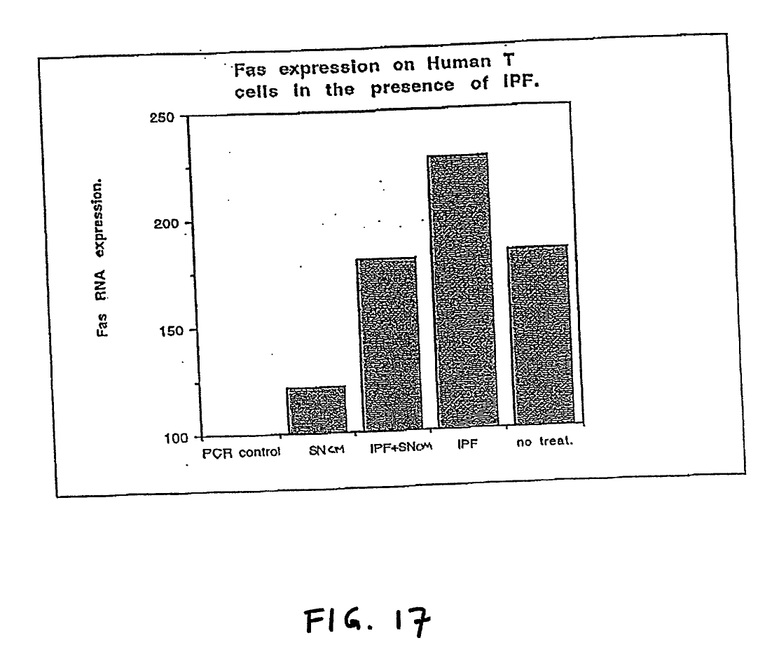

Following the incubation the cells were lysed and total RNA was extracted using a RNAzol kit supplied by Biotex Laboratories, Inc., Houston Tex. RNA concentration was evaluated and 1 μg RNA of each sample was reverse transcribed followed by Polymerase Chain Reaction (PCR) amplification using DNA primers derived from the human fas receptor gene, sense strand, 5′-AGATTATCGTCCAAAAGTGTTAATG-3′ (SEQ ID NO: 1); antisense strand, 5′-CAGAATTCGTTAGATCTGGATCCTTCCTC-3′ (SEQ ID NO: 2). The amplified products were visualized on a 2.5% agarose gel and quantified by densitometry. The results are presented in FIG. 17.

FIG. 17 shows that the human fas receptor gene transcript is upregulated in T cells in the presence of IPF. Since the fas receptor is known to be expressed in cells undergoing programmed cell death (apoptosis) and is involved in the process of apoptosis and since IPF induces expression of the fas receptor on T cells, IPF seems to play a role in maintaining immune privilege in the CNS by inducing apoptosis in immune cells.

The invention claimed and described herein is not to be limited in scope by the specific embodiments herein disclosed since these embodiments are intended as illustrations of several aspects of the invention. Indeed, various modifications of the invention in addition to those shown and described herein will become apparent to those skilled in the art from the foregoing description. Such modifications are also intended to fall within the scope of the appended claims.

A number of references are cited herein, the entire disclosures of which are incorporated herein, in their entirety, by reference.

What is claimed is:

1. A purified immune privilege factor which is obtainable by a process comprising:
(a) subjecting central nervous system tissue conditioned medium to gel filtration chromatography;
(b) collecting the fractions which inhibit macrophage migration in an in vitro assay;
(c) subjecting the fractions collected in (b) to reverse phase high pressure liquid chromatograph (HPLC);
(d) collecting the fractions which inhibit macrophage migration in an in vitro assay;
(e) subjecting the fractions collected in (d) to ion exchange column chromatography; and
(f) collecting the fractions which inhibit macrophage migration in an in vitro assay.

2. A composition comprising an immune privilege factor in accordance with claim 1.

3. The composition according to claim 2, further comprising a pharmaceutically acceptable carrier.

4. A purified immune privilege factor which:
(1) inhibits macrophage adhesion in an in vitro assay,
(2) is heat stable at 100° C., and
(3) is obtainable from optic nerve conditioned medium in gel filtration chromatography fractions corresponding to a molecular weight of about 350 Daltons, followed by elution from a reverse phase chromatography column in a fraction exhibiting inhibition of macrophage adhesion in an in vitro assay.

5. A purified immune privilege factor in accordance with claim 4 which has an amino acid composition which includes glutamic acid, serine and glycine.

6. A composition comprising an immune privilege factor in accordance with claim 4.

7. The composition according to claim 6, further comprising a pharmaceutically acceptable carrier.

8. A method for the inhibition of inflammation associated with a disease, condition or disorder of the mammalian central nervous system or the eye comprising applying an effective amount of an approximately 350 Dalton central nervous system derived heat stable immune privilege factor which inhibits macrophage migration and/or macrophage phagocytic activity.

9. The method according to claim 8, in which the disease, condition or disorder is blunt trauma, AIDS-related dementia complex, HIV-related encephalopathy, post-polio syndrome, multiple sclerosis, myelitis, encephalitis, meningitis, rheumatic fever, complications and side-effects due to neurosurgery, subacute sclerosing panencephalitis, Huntington's disease, Parkinson's disease, Devic's disease, Sydenham chorea, Alzheimer's disease, posterior uveitis, anterior uveitis, sympathetic ophthalmia, retinitis, cystoid macular edema, optic neuritis, proliferative vitreoretinophathy, retinitis pigmentosa or a complication and/or side-effect from transplantation surgery or treatment of Parkinson's disease.

10. The method according to claim 8, in which the factor is applied locally to a site in the central nervous system or eye by injection, local infusion, topical application or an implant.

11. The method according to claim 8, in which the factor is applied systemically by intravenous or intramuscular injection.

12. A method for the inhibition of inflammation associated with a disease, condition or disorder of the mammalian central nervous system or the eye comprising applying an effective amount of an immune privilege factor in accordance with claim 1.

13. The method according to claim 12, in which the disease, condition or disorder is blunt trauma, AIDS-related dementia complex, HIV-related encephalopathy, post-polio syndrome, multiple sclerosis, myelitis, encephalitis, meningitis, rheumatic fever, complications and side-effects due to neurosurgery, subacute sclerosing panencephalitis, Huntington's disease, Parkinson's disease, Devic's disease, Alzheimer's disease, Sydenham chorea, posterior uveitis, anterior uveitis, sympathetic ophthalmia, retinitis, cystoid macular edema, optic neuritis, proliferative vitreoretinophathy, retinitis pigmentosa or a complication and/or side-effect from transplantation surgery or treatment Parkinson's disease.

14. The method according to claim 12, in which the factor is applied locally to a site in the central nervous system by injection, local infusion, topical application or an implant.

15. The method according to claim 12, in which the factor is applied systemically by intravenous or intramuscular injection.

16. The method according to claim 12, in which the central nervous system tissue conditioned medium is optic nerve conditioned medium.

17. The method according to claim 12, in which the central nervous system tissue conditioned medium is brain tissue conditioned medium.

18. A method for the inhibition of inflammation associated with a disease, condition or disorder of the mammalian central nervous system or the eye comprising applying an effective amount of an immune privilege factor in accordance with claim 4.

19. A method for the inhibition of inflammation associated with a disease, condition or disorder of the mammalian central nervous system or the eye comprising applying an effective amount of an immune privilege factor in accordance with claim 5.


SEQUENCE LISTING (2 sequences)

SEQ ID NO: 1 - DNA, 25 bp, Artificial Sequence
  Description of Artificial Sequence primer
agattatcgtccaaaagtgttaatg

SEQ ID NO: 2 - DNA, 29 bp, Artificial Sequence
  Description of Artificial Sequence primer
cagaattcgttagatctggatccttcctc